(19) United States
(12) Patent Application Publication
(10) Pub. No.: US 2018/0085926 A1
(43) Pub. Date: Mar. 29, 2018

(54) Robot System And Method For Operating A Teleoperative Process

(71) Applicant: KUKA Roboter GmbH, Augsburg (DE)

(72) Inventor: Yevgen Kogan, Augsburg (DE)

(73) Assignee: KUKA Roboter GmbH, Augsburg (DE)

(21) Appl. No.: 15/559,199

(22) PCT Filed: Mar. 17, 2016
(86) PCT No.: PCT/EP2016/055851

§ 371 Date: Sep. 18, 2017

(30) Foreign Application Priority Data
Mar. 18, 2015 (DE) 10 2015 204 867.5

(51) Int. Cl. B25J 9/16; B25J 19/06; B25J 19/02; A61B 34/00; A61B 90/00

(52) U.S. Cl. CPC B25J 9/1689; B25J 9/1676; B25J 9/1682; B25J 19/06; A61B 34/35; B25J 19/026; A61B 34/77; A61B 90/361; G05B 2219/39114; B25J 19/023

(57) ABSTRACT

A system and a method for carrying out a teleoperative process, wherein an image recording device and a tool are used. The tool may be guided by a first manipulator, and a currently captured region of the image recording device is determined. Monitoring the position of the tool relative to the currently captured region of the image recording device makes it possible to prevent the tool from unintentionally leaving the captured region. The axes of the manipulator are provided with sensors for detecting the forces and/or torques acting on the axes.

### CROSS-REFERENCE

This application is a national phase application under 35 U.S.C. § 371 of International Patent Application No. PCT/EP2016/055851, filed Mar. 17, 2016 (pending), which claims the benefit of German Patent Application No. DE 10 2015 204 867.5 filed Mar. 18, 2015, the disclosures of which are incorporated by reference herein in their entirety.

### TECHNICAL FIELD

The present invention relates to a robot system and a method for carrying out a teleoperative process, such as, for example, for the robot-assisted machining of workpieces or the medical treatment of a patient, wherein a robot-guided tool and an image recording device are used.

### BACKGROUND

Telerobotics is a subfield of robotics that is concerned with the control of robots from a distance. Remote controlled manipulators are also referred to as teleoperators; and telerobotics is used in many industrial applications. Another application is medicine, where robot-assisted systems are operated teleoperatively, in particular, in surgery. For example, at the present time medical studies or treatments, based on image recording devices, such as ultrasound devices or endoscopic imaging devices, are considered to be standard practice in medicine. One example of a medical treatment of this type is minimally invasive surgery, where endoscopic instruments, such as biopsy needles, are used as tools, and where the use of these medical instruments inside a patient's body is monitored with image recording devices, such as a video endoscope. In such procedures the medical instrument has to stay in the region captured by the image recording device, so that the operator or, more specifically, the surgeon has a visual feedback of his work. In such systems, however, it can happen that a disadvantageous positioning of the image recording device or the instrument will result in the instrument moving out of the captured region, for example, out of the camera field of view of the video endoscope, during the operation. In this case the camera or rather the instrument has to be positioned in such a way that the instrument is visible again. However, it is not always obvious (from outside the patient's body) how the camera or rather the instrument has to be moved. Therefore, such a situation should be avoided, whenever possible. Similar problems exist in a number of other teleoperative processes.

The prior art discloses methods, in which an image recording device, i.e., an ultrasound head, is guided by means of a manipulator, in particular, a robot. For example, U.S. Pat. No. 7,753,851 discloses a known robot system, which has a probe that is mounted on the hand flange of the robot and that can be moved by the robot. Compared to the manual operation of the probe, the robot-assisted approach allows a particularly precise orientation of the probe.

From U.S. Pat. No. 6,236,875 B1 it is known to specify and monitor a predefined safe zone in the body of a patient (for example, an area surrounding a particular organ of the patient). The position of the medical instrument that is used for the procedure is also monitored; and the instrument is deactivated when it approaches the safe zone.

US 2013/0038707 A1 describes a method to offer a surgeon the option of a visual feedback by way of a video camera during a minimally invasive surgical procedure. In this case, too, the objective is to monitor the medical instrument that is used, in order to prevent the instrument from entering into a predefined safe zone in the body of the patient.

One drawback with some of the above methods is that the definition of safe zones requires an elaborate plan, in which, in particular, the respective safe zones have to be defined for each individual case. In addition, the patient's body has to be either fixed or tracked during surgery, so that the current position of the body corresponds to the predefined safe zones. In addition, it is also possible in these cases that the medical instrument leaves the captured region of the image recording device, thus, for example, the field of view of the camera that is used, since the image recording device and also the tool are usually movable. These disadvantages exist analogously in other, for example, industrial applications.

Therefore, the object of the present invention is to provide an improved system and method for operating a teleoperative process, such as, for example, for the robot-assisted medical treatment of a patient, in such a way that the drawbacks of the prior art can be avoided or minimized. In particular, an object of the present invention is to provide automatically suitable countermeasures, if a tool is threatening to leave the captured region of an image recording device.

The aforementioned and other engineering objects, which will become more apparent from the following detailed description, are achieved by means of the subject matter of the independent claims 1 and 7.

### SUMMARY

The present invention relates, in particular, to a robot system for a teleoperative process, in which robot system an image recording device and a tool are provided, wherein the tool is guided by a first manipulator. The manipulator is, in particular, a multi-axis articulated robot arm. In particular, the axes of the manipulator can be provided with sensors for detecting the forces and/or torques acting on the axes. In this context the system comprises a control unit that is configured to determine the currently captured region of the image recording device and the pose of the tool (such as, for example, the tip of a needle or a welding wire) relative to the image recording device and to perform an action, when the tool violates the limits of the currently captured region of the image recording device. The term “currently captured region” is defined as that region in a space that the image recording device can capture in its respective position and orientation. By knowing the pose of the tool relative to the image recording device, it is possible for the control unit to determine whether and how the tool is captured by the image recording device. For many applications it suffices if only a certain part of the tool, such as, for example, the tip of a tool that is not supposed to enter or leave a particular region, or any other part of the tool is captured. Therefore, the step of “determining the pose of the tool” is construed herein in the broad sense and also includes the determination of the pose of only a part of the tool. In principle, it is advantageously possible to define the limits of the currently captured region as a function of the application. For example, the quality of the visualization usually declines at the edge of the captured region. Therefore, depending upon the application, i.e., the required quality of visualization, it is possible to adjust the limit at which, for example, an alarm is emitted.

The sensors for detecting the forces and/or torques acting on the axes can be, for example, current and/or voltage sensors, which monitor the current/the voltage of the drives of the axes of the manipulator, in order to detect the forces and/or torques acting on the axes. Similarly the sensors may be resistive, capacitive and/or inductive force and/or torque sensors. Furthermore, piezoelectric sensors or other known sensors can be used that make it possible to detect forces and/or torques.

Preferably the action comprises the output of a warning and/or a shutdown or, more specifically, hard switching of the first manipulator and/or a deactivation of the tool. These actions can also be staggered depending on the situation. Hence, it is advantageous, for example, if an alarm signal is emitted, for example, by means of visual or auditory cues, in a first step, when the tool comes too close to the edge of the captured region. If the controller determines that after the warning the tool is still being guided closer to the edge of the captured region, then the tool, such as, for example, an electrotomy device or a welding electrode, is deactivated, for example, in a second step. The first manipulator is shut down only as a last safety measure. The term “shutdown” is defined herein as a subtype of the hard switching operation. During the hard switching operation the movements of the manipulator are rendered artificially more difficult, so that a user, for example, a surgeon, gets a tactile (haptic) feedback from a force feedback controller, with which he controls the manipulator. The hard switching operation may inhibit the movement of the manipulator in such a way that the manipulator is virtually stopped. Of course, a shutdown can also take place by activating the mechanical brakes of the manipulator or by means of any other appropriate measures.

Furthermore, the image recording device is guided preferably by a second manipulator, in particular, a multi-axis articulated robot arm. The first and second manipulators cooperate; and the control unit is advantageously able to retrieve data about the pose of both manipulators. A multi-axis articulated robot arm is preferred, because it offers a high degree of freedom and can guide the image recording device or the tool in a very flexible way.

Preferably the control unit is further configured to determine the currently captured region of the image recording device by means of the manipulator controller of the second manipulator. The control unit is configured, for example, to retrieve or process data about the position of the second manipulator and preferably also about the first manipulator. With this position data it is possible to calculate the position and orientation of the image recording device in a space. Since the physically capturable or rather field of view of the image recording device is known, it is relatively easy to determine the currently captured region of the image recording device.

It is particularly preferred that the control unit be further configured to determine the pose of the tool by means of the manipulator controller of the first manipulator. The manipulator controller provides, for example, the data about the position of the first manipulator. Since the arrangement or, more specifically, the mounting of the tool on the manipulator is predetermined, it is easy to find the exact pose of the tool in a space. It is particularly advantageous if two manipulators are used, and the control unit can access the data about the position of both manipulators. If the pose of the two manipulators relative to each other is known, a relationship that can be easily determined, then it is possible for the control unit to determine directly the relative pose of the tool to the image recording device and, thus, ultimately to the currently captured region of the image recording device.

Preferably the manipulators that are used (that is, the first manipulator and the second manipulator, when the image recording device is guided by a second manipulator) are multi-axis articulated robot arms, the axes of which are provided preferably with sensors for detecting the forces and/or torques acting on the axes. With the aid of sensors it is possible to define for each manipulator the force limits that said manipulator may not exceed, when said manipulator presses, for example, the image recording device against a patient's body.

Preferably the image recording device is an ultrasound probe or an endoscopic imaging device. In the case of an ultrasound probe it is typically guided along the patient's body, for which purpose the aforementioned articulated robot arms, equipped with sensors, are particularly suited, since the ultrasound probes work optimally only if they are guided with the correct contact force against the patient's body.

In principle, the control unit is preferably a stand-alone computer system, which is connected to the manipulator controller of the first and optionally the second manipulator. However, the tasks of the control unit can also be fulfilled by, for example, the manipulator controller of the first manipulator or, optionally, the second manipulator, provided that the latter has sufficient computing capacity.

The present invention also relates to a method for operating a teleoperative process, such as, for example, the robot-assisted medical treatment of a patient, in which method the pose of a tool (such as, for example, a medical instrument), which is guided by a first manipulator, in particular, a multi-axis articulated robot arm, is determined in one step. Then it is very easy to determine the pose, as stated above, since the current position data of the first manipulator are known from the manipulator controller. Furthermore, in the method the currently captured region of an image recording device, such as, for example, an ultrasound probe or a videoscope, is determined, which image recording device is used to provide a user (for example, a surgeon) a visualization of the pose of the tool, such as, for example, the visualization of the pose of the tip of the tool. If the image recording device is also guided by a (second) manipulator, then this determination is easily possible by means of the position data from the manipulator controller. It is also possible to provide the image recording device with, for example, markers. The markers on the image recording device are then detected, for example, by a suitable sensor, in order to be able to detect the pose of the marker in a space, and, in so doing, the position of the image recording device, since the offset between marker and image recording device is known.

In a further step the pose of the tool, such as, for example, the pose of the tip of a welding electrode, is determined relative to the image recording device, and a warning is emitted, and/or the first manipulator is shut down or, more specifically, is hard switched and/or the tool is deactivated, when the tool violates the limits of the currently captured region of the image recording device. As explained above, these limits can be defined as a function of the specific application requirement. When high demands are made on the quality of visualization, a warning signal can be emitted to the user, for example, at an early stage, before the tool has reached the edge of the physically capturable region of the image recording device, in which region an image can still be produced, but its quality may no longer be sufficient.

Basically it is also preferred in the method that the image recording device be guided by a second manipulator, in particular, a multi-axis articulated robot arm. This allows advantageously that the currently captured region of the image recording device can be determined by means of the manipulator controller of the second manipulator. The manipulator controller has accurate and current data about the position of the second manipulator; and since the relative pose of the image recording device to the second manipulator or, more specifically, to the coordinate system of the manipulator is known, it is, therefore, easy to calculate the captured region of the image recording device.

Furthermore, the pose of the tool is determined preferably by means of the manipulator controller of the first manipulator.

For the aforementioned reasons it is further preferred to use multi-axis articulated robot arms in the method according to the invention, wherein the axes of said articulated robot arms are provided with sensors for detecting the forces and/or torques acting on the axes.

### BRIEF DESCRIPTION OF THE DRAWINGS

The present invention is described below with respect to one non-restrictive exemplary embodiment.

FIG. 1 shows in schematic form an inventive system for the robot-assisted treatment of a patient.

### DETAILED DESCRIPTION

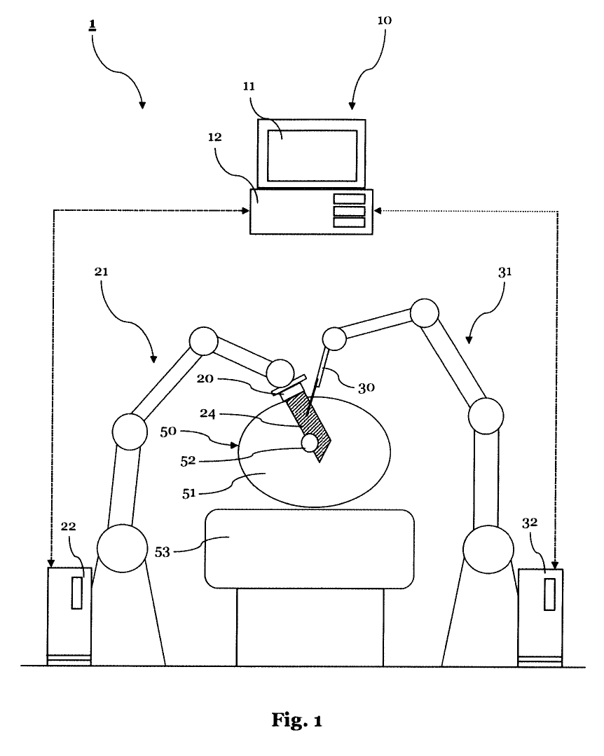

FIG. 1 shows in schematic form and by way of example an inventive system 1 for the robot-assisted treatment of a patient 50. It is clear to those skilled in the art that the principles, described herein, may be applied, of course, to any type of teleoperation, in particular, to teleoperations in an industrial environment. The system comprises a control unit 10, which comprises a computer 12 and a screen 11. The patient 50 lies on an operating table 53; and in the drawing shown, the reference numeral 51 is intended to indicate a cross sectional view through the neck of the patient 50. In the neck 51 there is a target point 52, such as, for example, a tumor or the like, that is to be examined or, more specifically, is to be treated. The tool that is to be used in the treatment is a surgical instrument, i.e., a biopsy needle 30. The biopsy needle 30 is guided by a first manipulator 31, which is a multi-axis articulated robot arm 31 in the illustrated case. The articulated robot arm 31 is assigned a manipulator controller 32, which is connected to the computer 12 of the control unit 10, as indicated by the dashed arrows. The biopsy needle 30 is to be guided to the target point 52. In order to make it easier for the surgeon to guide the biopsy needle 30 or to make it possible at all, an image recording device in the form of an ultrasound probe 20 is used. The ultrasound probe 20 is guided by a second manipulator 21, which is also a multi-axis articulated robot arm and which is assigned a manipulator controller 22. The manipulator controller 22 and also the ultrasound probe 20 are connected to the control unit 10, as indicated by the dashed arrows.

The articulated robot arm 21 carries and moves the ultrasound probe 20. The ultrasound probe 20 is pressed by the articulated robot arm 21 against the body of the patient 50 in order to make ultrasound images of the inside of the patient's body. The ultrasound images are transmitted to the control unit 10 or, more specifically, the associated computer 12, processed in the computer 12 and then displayed on the screen 11. The reference numeral 24 is intended to indicate the currently captured region of the ultrasound probe 20 (i.e., the image plane (sound plane) of the ultrasound probe). The image plane or sound plane of the probe is typically only a few millimeters thick, so that the probe has to be aligned very precisely in order to deliver informative images. The alignment of the probe and the pressing of the probe is carried out by the manipulator or, more specifically, by the articulated robot arm 21, so that a surgeon is relieved of these tasks. To this end it is advantageous for the articulated robot arm 21 to be provided with force sensors and to work in a closed loop force control, so that the articulated robot arm presses the ultrasound probe 20 with a defined force against the skin surface of the patient 50.

Since the pose of the ultrasound probe 20 is fixed, based on the current position of the manipulator, or can be calculated from it, and since the contour and the orientation of the captured region 24 are also known, it is possible to calculate precisely where the captured region 24 is located in the space.

In FIG. 1 the tip of the biopsy needle 30 is inside the currently captured region 24, so that the surgeon can track the movement of the tip through the body of the patient 50 on the screen 11 and can guide the biopsy needle 20 accordingly in a target-oriented manner to the target point 52. The position and orientation of the biopsy needle is known precisely due to the robot's position and the pose of the manipulator 31 or can be accurately determined therefrom. Since the control unit 10 knows the respective position and pose of the two manipulators 21 and 31 from the two manipulator controllers 22 and 32 or can calculate said position and pose with the aid of (i.e., by means of) the manipulator controllers, the control unit 10 can determine the pose of the biopsy needle 30 relative to the ultrasound probe 20 and, thus, also the relative-pose of, for example, the tip of the biopsy needle 30 to the currently captured region 24. This allows the control unit to determine if the biopsy needle 30 has violated the limits of the captured region 24 of the ultrasound probe 20. If such a violation of the limits is determined, then a corresponding warning can be emitted, for example, on the screen 11, or the manipulator 31 is hard switched.

While the present invention has been illustrated by a description of various embodiments, and while these embodiments have been described in considerable detail, it is not intended to restrict or in any way limit the scope of the appended claims to such detail. The various features shown and described herein may be used alone or in any combination. Additional advantages and modifications will readily appear to those skilled in the art. The invention in its broader aspects is therefore not limited to the specific details, representative apparatus and method, and illustrative example shown and described. Accordingly, departures may be made from such details without departing from the spirit and scope of the general inventive concept.

### LIST OF REFERENCE NUMERALS

1 system
10 control unit
11 screen
12 computer
20 image recording device (ultrasound probe)
21, 31 manipulators (articulated robot arm)
22, 32 manipulator controllers
24 currently captured region (sound plane)
30 tool (biopsy needle)
50 patient
51 cross section through the neck
52 target point
53 operating table

What is claimed is:

1-12. (canceled)

13. A robot system for a teleoperative process, comprising:
an image recording device;
a tool guided by a first manipulator, wherein the axes of the manipulator are provided with sensors for detecting at least one of forces or torques acting on the axes; and
a control unit configured to:
(a) determine a currently captured region of the image recording device,
(b) determine a pose of the tool relative to the image recording device, and
(c) perform an action when the tool violates limits of the currently captured region of the image recording device.

14. The robot system of claim 13, wherein the first manipulator is a multi-axis articulated robot arm.

15. The robot system of claim 13, wherein the action performed by the control unit comprises at least one of the output of a warning, a shutdown, hard switching of the first manipulator, or a deactivation of the tool.

16. The robot system of claim 13, wherein the image recording device is guided by a second manipulator.

17. The robot system of claim 16, wherein the second manipulator is a multi-axis articulated robot arm.

18. The robot system of claim 16, wherein the control unit is further configured to determine the currently captured region of the image recording device using a manipulator controller of the second manipulator.

19. The robot system of claim 13, wherein the control unit is further configured to determine the position of the tool using a manipulator controller of the first manipulator.

20. The robot system of claim 13, wherein the image recording device is an ultrasound probe or an endoscopic imaging device.

21. The robot system of claim 13, wherein the control unit determines the pose of the tool relative to the image capturing device by determining the pose of only a part of the tool.

22. A method for operating a teleoperative process, comprising:
determining the pose of a tool guided by a first manipulator, wherein the axes of the manipulator are provided with sensors for detecting at least one of forces or torques acting on the axes;
determining a currently captured region of an image recording device that provides a user with a visualization of the pose of the tool;
determining the pose of the tool relative to the image recording device; and
when the tool violates limits of the currently captured region of the image recording device, then at least one of emitting a warning, shutting down, hard switching the first manipulator, or deactivating the tool.

23. The method of claim 22, wherein the first manipulator is a multi-axis articulated robot arm.

24. The method of claim 22, wherein the image recording device is guided by a second manipulator.

25. The method of claim 24, wherein the second manipulator is a multi-axis articulated robot arm.

26. The method of claim 24, wherein the currently captured region of the image recording device is determined using a manipulator controller of the second manipulator.

27. The method of claim 22, wherein the pose of the tool is determined using a manipulator controller of the first manipulator.

28. The method of claim 22, wherein the image recording device is an ultrasound probe or an endoscopic imaging device.

29. The method of claim 22, wherein determining the pose of the tool relative to the image capturing device comprises determining the pose of only a part of the tool.
